(19) United States
(12) Patent Application Publication
(10) Pub. No.: US 2009/0233292 A1
(43) Pub. Date: Sep. 17, 2009

(54) Method and kit for identification of genetic polymorphisms

(76) Inventors: Daniele Podini, Washington, DC (US); Moses Schanfield, Silver Spring, MD (US); Tahnee Nelson, San Francisco, CA (US); Melinda Hung, Alexandria, VA (US)

Correspondence Address: BLANK ROME LLP, WATERGATE, 600 NEW HAMPSHIRE AVENUE, N.W., WASHINGTON, DC, 20037, US

(21) Appl. No.: 12/325,908

(22) Filed: Dec. 1, 2008

Related U.S. Application Data
(60) Provisional application No. 60/991,247, filed on Nov. 30, 2007.

(51) Int. Cl. C12Q 1/68

(52) U.S. Cl. 435/6

(57) ABSTRACT

Provided herein are primer sets, kits, and methods for identifying mtDNA polymorphisms in a sample. In one embodiment, the primer sets, kits and methods are directed to identifying global haplogroups (“Global”). In another embodiment, the primer sets, kits, and methods are directed to identifying specific European haplogroups (“European”).

### CROSS REFERENCE TO RELATED PATENT APPLICATIONS

This application claims the right of priority under 35 U.S.C. 119(e) and is entitled to the benefit of the earlier filing date of U.S. Provisional 60/991,247 filed 30 Nov. 2007.

### STATEMENT REGARDING FEDERALLY SPONSORED RESEARCH OR DEVELOPMENT

Not applicable.

### NAMES OF PARTIES TO A JOINT RESEARCH AGREEMENT

The George Washington University

The Armed Forces DNA Identification Laboratory, Rockville, Md., USA

### REFERENCE TO A SEQUENCE LISTING

A table or a computer list appendix on a compact disc [ ] is /[ ] is not included herein and the material on the disc, if any, is incorporated-by-reference herein.

### BACKGROUND OF THE INVENTION

This invention relates to analysis of mitochondrial DNA, and in particular, to methods and kits for identifying certain polymorphisms related to maternal ancestry in a simple, robust, and rapid process.

Interest in identifying mtDNA polymorphisms spans from identifying the remains of people killed in wartime or from accidents to identifying DNA evidence from crime scenes to identifying a person's geographic ancestry or lineage. However, current procedures for performing this identification are limited, such as by being too slow or complex, or by the inability to analyze highly degraded samples.

Accordingly, the present inventive subject matter is provided to address these and other needs arising in the forensic arts.

Mitochondrial DNA (mtDNA) is a 16,569 bp circular molecule present, on average, in 500 copies per cell (1). MtDNA analysis is utilized in several areas of science including, but not limited to, anthropology, evolutionary studies, and forensic science (2-5). The high copy number, and possibly the cellular location and molecular features of mtDNA, allow for increased recovery, thus providing a distinct advantage over nuclear DNA when working with highly compromised samples (6). The maternal inheritance and high mutation rate are characteristics extremely useful for evolutionary studies (7,8); in fact, mtDNA has been used to resolve evolutionary questions related to extinct species and to human migrations throughout the continents (9-12). The field of forensic science also relies upon mtDNA to identify missing persons, locate maternal relatives, identify victims in mass disasters, and, in some situations, include an individual at a crime scene (13-19).

Early studies of the mtDNA genome revealed patterns of variation that were linked to geographic regions. Individuals with the same sequence variations were clustered into haplogroups defined by mutations at particular nucleotide positions (20-27). A closer examination of the mtDNA genomes of various populations led to the following assumptions: 1) several of the mtDNA mutations are highly correlated with the ethnic and geographic origin of the individual, 2) all mutations originated from a single mtDNA tree, and 3) the greatest variation and deepest root of the mtDNA tree is present in the African population. Furthermore, a calculation of the variation between mtDNA haplogroups demonstrated that 35% of the mutations were continent-specific, and therefore useful indicators of geographic origin (24,25,28-31).

Before the advent of modern sequencing methods, the primary approach to identifying polymorphic sites throughout the mtDNA coding region was restriction fragment length polymorphism (RFLP) analysis. While this methodology is still utilized in certain contexts, direct sequencing of the mtDNA molecule is rapidly gaining acceptance as the method of choice for haplogroup typing (20,23,30,32-35). Single base primer extension, also known as minisequencing, is an example of a direct sequencing technique that is currently utilized for mtDNA haplogroup typing (36-42). This methodology, described in detail by Fiorentino et al (43), offers several advantages to the investigator over RFLP and conventional sequence analysis methods—small amplicons (<150 bp), increased sensitivity and robustness, and multiplexing capability. Multiplexing capability is particularly important, especially in regard to forensic DNA analysis, as it reduces sample consumption while increasing throughput of sample processing and data analysis. Increased sensitivity allows for improved amplification success with DNA samples that contain limited starting template. Additionally, the possibility for high throughput processing can aid in population screening studies in situations where numerous samples need to be typed (44). This is particularly true in mass disaster or other mass screening situations, where a simple and rapid population screening tool that consumes little extract could effectively direct subsequent identification testing. In these situations, coding region sequencing would be expensive and time-consuming, and the subsequent data analysis a lengthy, burdensome, and potentially error-prone process (42,45-48). Furthermore, the possibility of obtaining interpretable results from poor quality polymerase chain reaction (PCR) products while simultaneously typing several polymorphisms throughout the mtDNA genome make it a more feasible method than conventional PCR fragment sequence analysis, especially in forensic cases and anthropological studies involving highly degraded or otherwise compromised human remains (16,36-38,49,50).

### SUMMARY OF THE INVENTION

Provided herein are primer sets, kits, and methods for identifying mtDNA polymorphisms in a sample. In one embodiment, the primer sets, kits and methods are directed to identifying global haplogroups (“Global”). In another embodiment, the primer sets, kits, and methods are directed to identifying specific European haplogroups (“European”). The primer sets are unique and comprise one PCR primer set and one single base primer extension, or minisequencing, primer set. Accordingly, the Global primer set comprises one PCR and one minisequencing primer set, and the European primer set comprises one PCR and one minisequencing primer set.

Provided herein are primer sets, kits, and methods for identifying mtDNA polymorphisms in a sample. In one embodiment, the primer sets, kits and methods are directed to identifying global haplogroups (“Global”). In another embodiment, the primer sets, kits, and methods are directed to identifying specific European haplogroups (“European”).

The primer sets are unique and comprise one PCR primer set and one minisequencing primer set. Accordingly, the Global primer set comprises one PCR and one minisequencing primer set, and the European primer set also comprises one PCR and one minisequencing primer set.

In a preferred embodiment, provided herein is a method for identifying polymorphisms in a sample of human mitochondrial DNA, comprising: (i) obtaining the mitochondrial DNA from the sample; (ii) screening the mitochondrial DNA using a PCR primer set and a minisequencing primer set, wherein the primer sets are directed to haplogroups A, B, C, D, E, F, G, H, I, L, M, N, and X, said PCR primer set comprising primers described in Table 2 (SEQ ID NOS 1-22, respectively, in order of appearance) and said minisequencing primer set comprising primers described in Table 3 (SEQ ID NOS 23-34, respectively, in order of appearance).

In a preferred embodiment, is provided herein a PCR primer set, comprising the primers described in Table 2 (SEQ ID NOS 1-22, respectively, in order of appearance).

In a preferred embodiment, also provided is a minisequencing primer set, comprising the primers described in Table 3 (SEQ ID NOS 23-34, respectively, in order of appearance).

In yet another preferred embodiment, a kit is provided for performing the methods of the present invention.

In a preferred embodiment, a method is provided for identifying polymorphisms in a sample of human mitochondrial DNA, comprising:

obtaining the mitochondrial DNA from the sample;

screening the mitochondrial DNA using a PCR primer set and a minisequencing primer set, wherein the primer sets are directed to 16 SNPs that include the diagnostic polymorphic sites for the European haplogroups H, J, K, T, U, V, and W, said PCR primer set comprising primers described in Table 2.1 (SEQ ID NOS 35-45, 16, 46-61, respectively, in order of appearance) and said minisequencing primer set comprising primers described in Table 2.2 (SEQ ID NOS 62-76, respectively, in order of appearance).

In a preferred embodiment, a PCR primer set is provided, comprising the primers described in Table 2.1 (SEQ ID NOS 35-45, 16, 46-61, respectively, in order of appearance).

In a preferred embodiment, a minisequencing primer set is provided, comprising the primers described in Table 2.2 (SEQ ID NOS 62-76, respectively, in order of appearance).

In a further preferred embodiment, a kit is provided for performing the methods of the present invention.

### BRIEF DESCRIPTION OF THE FIGURES

FIG. 1: (Nelson) Visual representation of the diagnostic polymorphisms used to identify the haplogroup and ancestry of an individual using the SNP assay.

FIG. 2: (Nelson) Human femur (sample 1) allegedly collected near a 1945 plane crash site in the Philippine Islands.

FIG. 3: Electropherogram representing macrohaplogroup N and Cambridge reference sequence haplogroup H are included to demonstrate the ability to detect polymorphic sequences. The X-axis represents the size (bp) of the primer with the incorporated nucleotide, while the Y-axis corresponds to the relative fluorescent unit (RFU) of the peak. Each fluorescent dye corresponds to a different nucleotide where blue represents G, green represents A, yellow (depicted here as black for better visual contrast) represents C, and red represents T.

FIG. 4: Electropherogram obtained from the mtDNA extracted from a femur recovered from a WWII plane crash in the Philippine Islands shown in FIG. 2. The first peak is blue (G) which represents a C to G base change caused by the 8272-8280 9 bp deletion that defines Haplogroup B.

FIG. 5: Haplogroup H and Sub-haplogroups H1-H15.

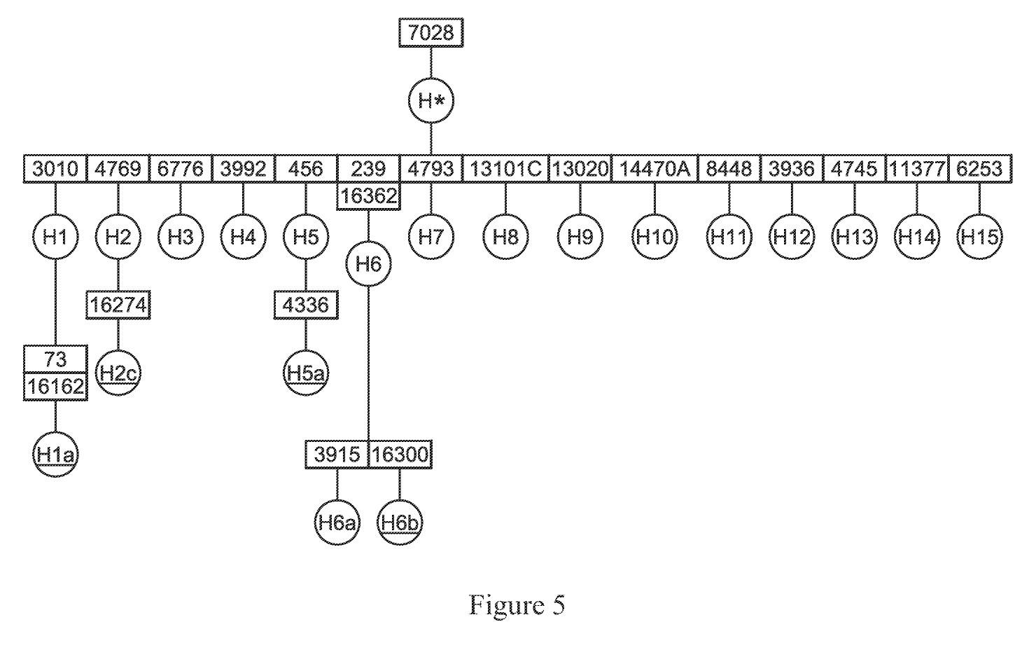

FIG. 6: Divergence of Major European Haplogroup

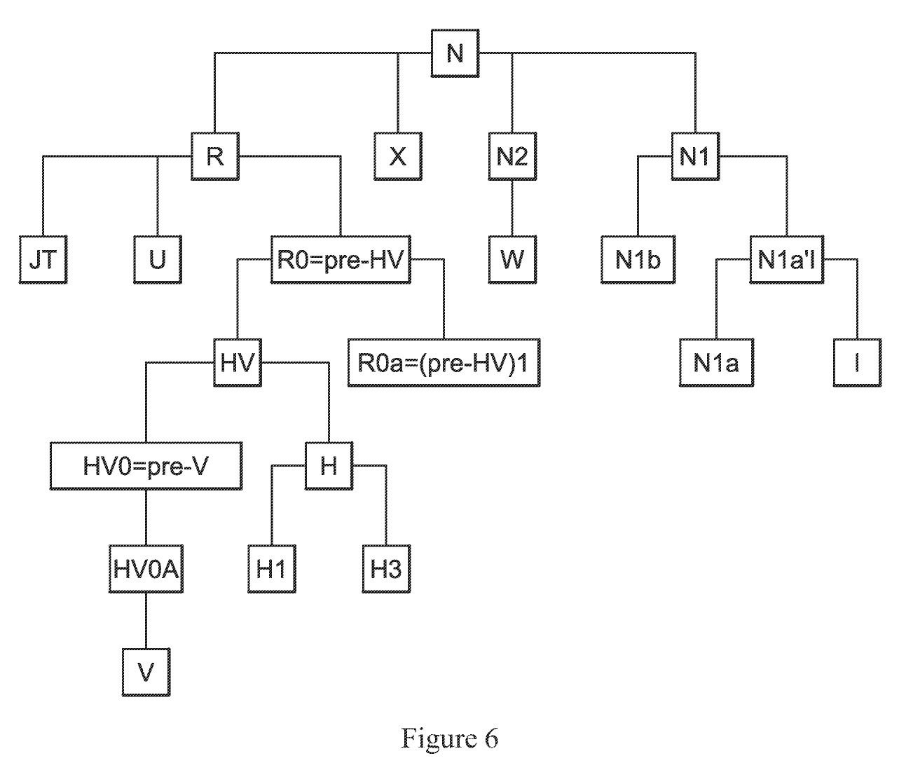

FIG. 7: Electropherogram of haplogroup H sample

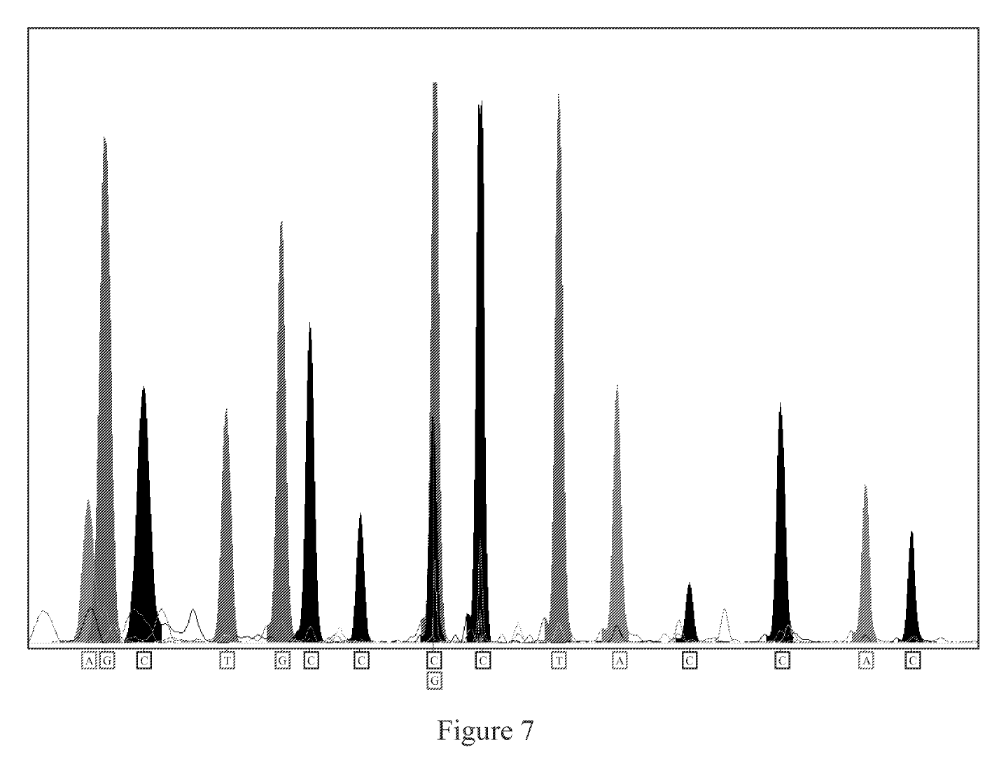

TABLE 1. Expected single nucleotide polymorphisms typing results and inferred ancestries for each haplogroup represented by the assay. Bases in bold are different than the Cambridge reference sequence (haplogroup H) and are used to differentiate haplogroup status.

TABLE 2. Global PCR primer set. (SEQ ID NOS 1-22, respectively, in order of appearance). Nucleotide position of the polymorphism, oligonucleotide sequence (5′→3′), primer orientation, primer length (bp), amplicon length (bp), Tm (° C.), GC content (%), and final concentration (nM) for each primer used during polymerase chain reaction amplification. The 10398 and 10400 polymorphisms are located on the same amplicon.

TABLE 3. Global minisequencing primer set. (SEQ ID NOS 23-34, respectively, in order of appearance). Nucleotide position of the polymorphism, sequence of the single base extension primer including polymeric T-stretch (5′→3′), orientation of the primer, primer length (bp), Tm of primer (° C.), GC content of primer (%), final concentration (nM) of each minisequencing primer, and the expected base substitution observed at the polymorphic site. For the single base extension primers designed in the reverse orientation the recorded base substitution remains in the forward orientation to facilitate interpretation.

TABLE 2.1 European PCR primer set. (SEQ ID NOS 35-45, 16, 46-61, respectively, in order of appearance). This table lists the sequence of the forward and reverse primers used for PCR amplification of the mtDNA SNP region. Also included are primer length, amplicon length, annealing temperature (Tm), and GC content.

TABLE 2.2. European minisequencing primer set. (SEQ ID NOS 62-76, respectively, in order of appearance). The bases listed in red are the actual primer sequence and the T in parentheses is preceded by a number indicating how many T's are in the poly-T tail for that primer. The locus is listed with the actual base change and whether the primer is forard or reverse: consequently, the observed base change has also been listed according to observed peak color for that polymorphism.

TABLE 4. Single nucleotide polymorphisms (SNP) typing results for Armed Forces DNA Identification Laboratory samples with a control region (CR) haplogroup assignment.

TABLE 5. Single nucleotide polymorphisms (SNP) typing results for Armed Forces DNA Identification Laboratory samples without a conclusive control region (CR) haplogroup assignment. Twenty one Armed Forces DNA Identification Laboratory samples with no conclusive haplogroup determined by CR sequence data were SNP typed. Samples that were identified as “D or G” haplogroups by CR sequencing are noted.

TABLE 6. Results from the analysis of 31 samples of known self-defined ancestral origin.

### DETAILED DESCRIPTION OF THE INVENTION

### Definitions

The term “SNP” is an acronym for Single Nucleotide Polymorphism, and refers to when a single nucleotide (building block of DNA) is replaced with another.

The term “oligonucleotide” as used herein is defined as a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “multiplex” refers to simultaneous testing within a single reaction.

The term “mitochondrial DNA” refers to the DNA usually located within cellular organelles called mitochondria, but which can, in forensic analysis, be found and tested well after cell death.

The term “PCR” is an acronym for polymerase chain reaction and refers to a method of exponentially amplifying a fragment of DNA to facilitate detection.

The term “primer set” refers to a set of primers, viz. nucleic acid strands and related synthetic primer having appropriately similar or equivalent functionality.

The term “minisequencing” refers to a method also known as the single base primer extension reaction, where a DNA polymerase is used specifically to extend a primer that anneals immediately adjacent to the nucleotide position to be analyzed with a single labeled nucleoside triphosphate complementary to the nucleotide at the variant site. This reaction allows highly specific detection of point mutations and single nucleotide polymorphisms (SNPs). Because all SNPs can be analyzed with high specificity at the same reaction conditions, multiplex high-throughput genotyping assays and PCR-based analysis are provided. The methods herein are not intended to be limited only to traditional gel-based formats, but also include multiplex detection on microarrays that have been developed and applied to minisequencing-based assays.

The term “nucleotide” refers to the purine, & pyrimidine ribonucleotides which are the structural units of DNA, RNA, and cofactors.

The term “haplogroup” refers to mitochondrial DNA haplogroups, viz. a group of haplotypes, with a common ancestor.

The term “primer” refers to an oligonucleotide which is capable of acting as a point of initiation of synthesis when placed under conditions in which primer extension is initiated. An oligonucleotide “primer” may occur naturally, as in a purified restriction digest or may be produced synthetically.

The term “sample” is used in a broad sense herein and is intended to include a wide range of biological materials as well as compositions derived or extracted from such biological materials. Exemplary samples include whole blood; red blood cells; white blood cells; buffy coat; hair; nails and cuticle material; swabs, including but not limited to buccal swabs, throat swabs, vaginal swabs, urethral swabs, cervical swabs, throat swabs, rectal swabs, lesion swabs, abcess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; serum; pulmonary lavage; lung aspirates; and tissues, including but not limited to, liver, spleen, kidney, lung, intestine, brain, heart, muscle, pancreas, biopsy material, and the like. The skilled artisan will appreciate that lysates, extracts, or material obtained from any of the above exemplary biological samples are also within the scope of the invention. Tissue culture cells, including explanted material, primary cells, secondary cell lines, and the like, as well as lysates, extracts, or materials obtained from any cells, are also within the meaning of the term biological sample as used herein. Microorganisms and viruses that may be present on or in a sample are also within the scope of the invention. Materials obtained from forensic settings are also within the intended meaning of the term sample.

Global Haplogroups Assay

Mitochondrial SNP Sites Selected for Assay—Global Haplogroups

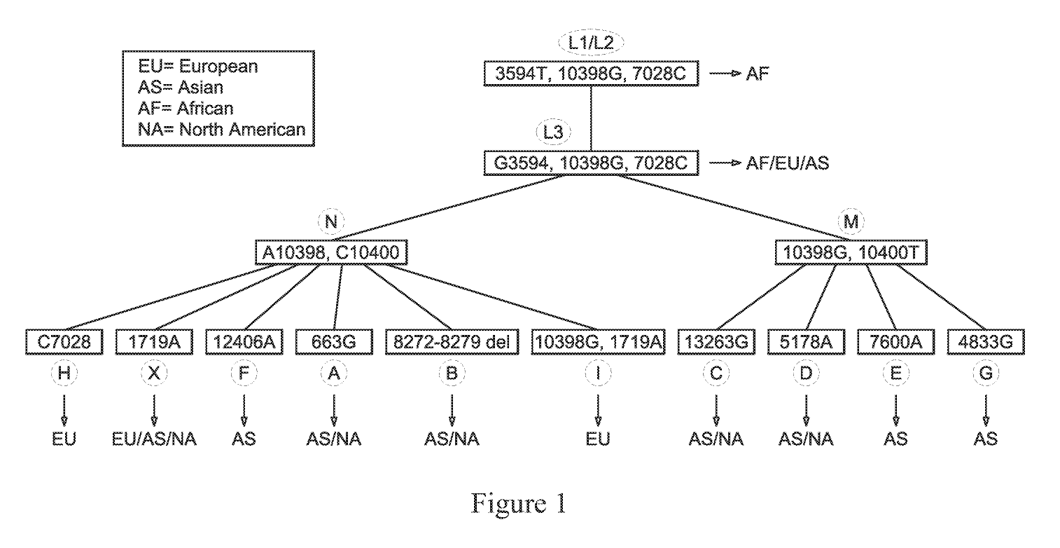

A review of the relevant literature (20-27, 33, 34) which collectively utilized 2500 fully sequenced mtDNA coding regions to generate haplogroup phylogenies, was the foundation for the selection of the twelve SNPs included in this assay. The intention was to maximize the number of haplogroups that could be identified using a specific set of polymorphisms (FIG. 1). Table 1 summarizes the expected results from each haplogroup typed by the assay. The SNPs were also selected for their ability to discriminate among major ancestral lineages (Europeans, Africans, Asians, and Native Americans) by examining only the coding region of the mitochondrial genome (24, 25, 28-31).

Primer Design—Global Haplogroups

Amplification and minisequencing primers utilized in the assay were designed using the Primer Express Version 2.0 software. Primers with comparable Tm and GC content properties and similar primer lengths were selected. Primer specificity was tested using the National Center for Biotechnology Information (NCBI) nucleotide BLAST search to eliminate the possibility of non-specific products during PCR. The PCR primers were designed to satisfy three criteria—1) must flank the desired SNP site, 2) must produce an amplicon no greater than 110 bp in length, and 3) the amplicon must retain the minisequencing primer annealing site for proper single base extension reaction (Table 2). The single base extension primers were designed one base contiguous to the polymorphic site of interest in either the forward or reverse orientation. Additionally, variable length polymeric-T tails were added the 5′ end of the primer in order to separate the products during electrophoresis (Table 3).

Multiplex PCR

The simultaneous amplification of the twelve amplicons containing the polymorphic sites of interest was carried out in a total volume of 50 μL. The reaction was conducted using the following reagents and concentrations: 0.05 U/μL of AmpliTaq Gold® DNA polymerase Applied Biosystems, Foster City, Calif., USA), 1× GeneAmp® PCR Gold Buffer (Applied Biosystems), 2 mM MgCl2 (Applied Biosystems), 200 μM each dNTP (Roche, Mannheim, Germany), 2 μL DNA extract, and 7.6 μL of DNA grade dH2O. The genomic DNA concentration of the samples varied between 0.2 to 1 ng/μL. The final concentrations of the PCR primers are listed in Table 2.

Thermocycling conditions followed a “reverse touch down” program adapted for single base extension assays by Vallone et al (35). The conditions were as follows: 95° C. for 11 minutes, 3 cycles of 95° C. for 30 seconds, 50° C. for 55 seconds, 72° C. for 30 seconds, 19 cycles of 95° C. for 30 seconds, 50° C. for 55 seconds +0.2° C. per cycle, 72° C. for 30 seconds, 11 cycles of 95° C. for 30 seconds, 55° C. for 55 seconds, 72° C. for 30 seconds, 72° C. for 7 minutes, and storage at 4° C. PCR amplification was carried out using GeneAmp® PCR System 9700 thermocyclers (Applied Biosystems), followed by agarose gel electrophoresis of the PCR product to verify that proper amplification occurred. Purification of 5 μL of PCR product was performed using 10 U of Exonuclease I (EXO) (USB, Cleveland, Ohio, USA) and 1 U of Shrimp Alkaline Phosphatase (SAP) (Roche Diagnostics Corporation, Indianapolis, Ind., USA) enzymes in a final volume of 7 μL. The thermocycling conditions for EXO-SAP purification were as follows: 70 minutes at 37° C. followed by 20 minutes at 72° C.

Single Base Extension of the Twelve Polymorphic Sites—Global Haplogroups

Single base primer extension was carried out using 2 μL of the SNaPshot® Ready Reaction Mix (Applied Biosystems), 2 μL purified PCR product, and 10 μL minisequencing primer mix in a 12 μL final volume. The final concentrations of the single base extension primers are listed in Table 3. The thermocycling conditions were as follows: 25 cycles of 95° C. for 10 seconds, 55° C. for 5 seconds, 60° C. for 30 seconds, and storage at 4° C. Samples were then purified with 1 unit of SAP for 70 minutes at 37° C., and 20 minutes at 72° C.

The SNP multiplex panel was developed in the laboratories of the Department of Forensic Sciences at The George Washington University using the ABI Prism® 310 Genetic Analyzer. Purified minisequencing products were prepared for capillary electrophoresis on the ABI Prism® 310 Genetic Analyzer by adding 1 μL of product to a 0.5 mL tube containing 11.85 μL of HIDI Formamide (Applied Biosystems) and 0.15 μL of Genescan-120 LIZ Size Standard (Applied Biosystems). The samples were denatured at 95° C. for 3 minutes and cooled at 4° C. The samples were then loaded on the ABI Prism® 310 Genetic Analyzer using a 47 cm×50 μM capillary filled with denaturing performance optimized polymer POP-4 (Applied Biosystems). The run parameters on ABI Prism® 310 Collection version 2.5 5-Dye Chemistry Software were as follows: 5-dye chemistry set up with module GS STR POP4 (1 mL) E5, 5-second injection time, 15 kV injection and run voltage, 60° C. run temperature, and a 15-minute run time. The data was then analyzed using the Genescan Version 3.7 software (Applied Biosystems).

Additional testing conducted at the Armed Forces DNA Identification Laboratory (AFDIL) was carried out using the ABI Prism® 3130 Genetic Analyzer utilizing the run parameters described in Vallone et al (35). Analysis was conducted using the Genemapper software (Applied Biosystems) with a custom panel and bin set to facilitate results interpretation. In addition, AFDIL results were analyzed with a custom-designed Excel macro that enabled automatic haplogroup typing from GeneMapper export files.

DNA samples

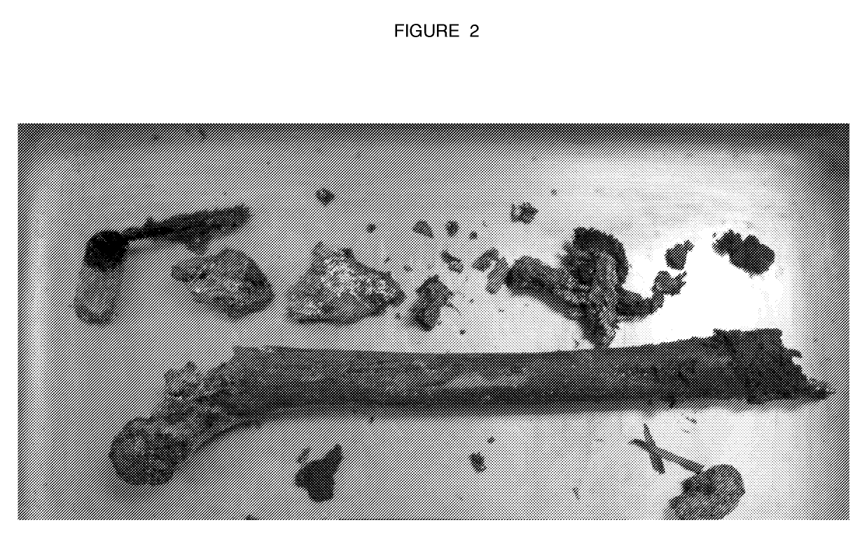

In all, 147 samples were analyzed to evaluate the ability of the assay to properly assign haplogroups. The set of DNA samples tested included 20 extracts from individuals previously typed using RFLP analysis (data from Dr Theodore Schurr, Pennsylvania State University), 73 samples for which the haplogroup was inferred based on the control region (CR) sequence data (Armed Forces DNA Identification Laboratory, Rockville, Md., USA, unpublished data), 21 AFDIL samples for which the CR-inferred haplogroup was inconclusive, and 31 extracts with no haplogroup assigned but of known ancestral origin (11 Europeans, 9 Africans, and 11 Asians). To evaluate the ability of the assay to obtain results at low concentrations, progressive dilutions, ranging from 1 to 0.007 ng of genomic DNA, were tested with the multiplex SNP panel. Additionally, to evaluate the assay's potential application in the forensic field, analysis of two extracts obtained from World War II era skeletal remains was conducted at the AFDIL. Sample 1 was a human femur whose potential origin was an American soldier killed in 1945 in a plane crash on Negros Island, Philippines (FIG. 2). Sample 2 was a human femur from one of four men lost in a 1943 plane crash in New Guinea. Both samples yielded no genomic DNA when quantitated with real time PCR.

Results

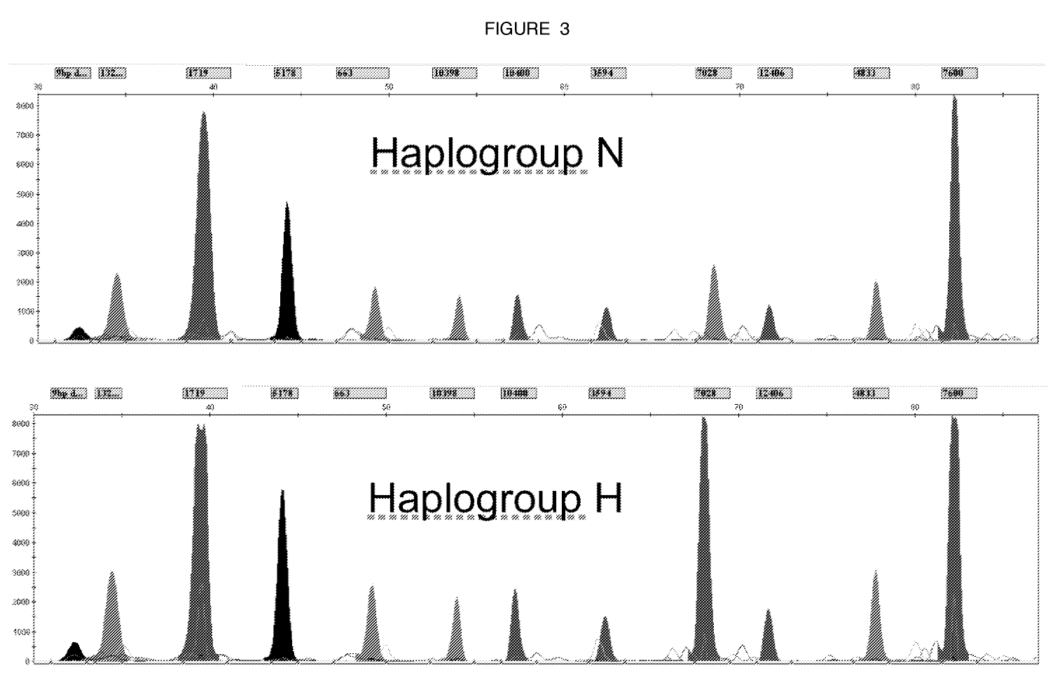

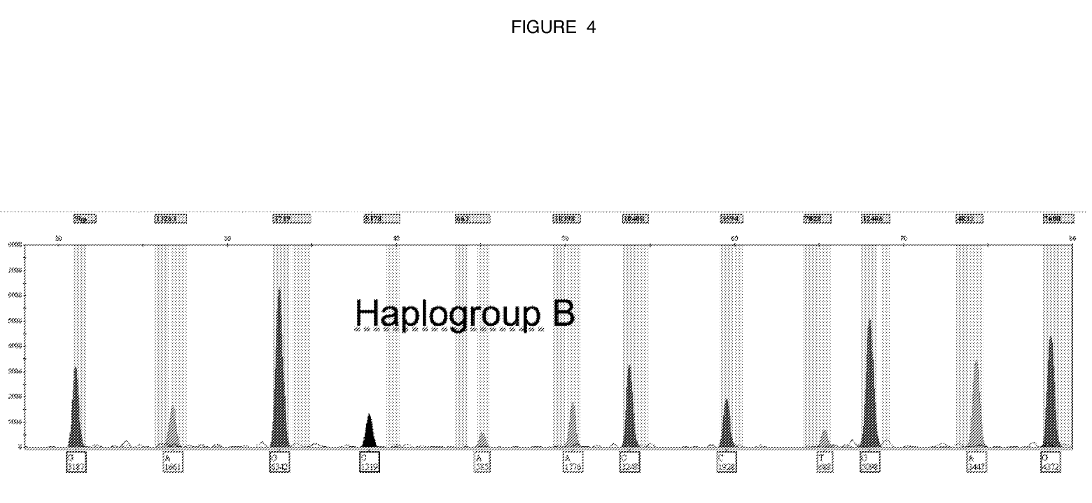

The order of migration of the primers and possible nucleotides incorporated is as follows (“F” and “R” represent the forward or reverse orientation of the primers, respectively): 9 bp deletion (C-G) F, 13263 (A-G) F, 1719 (G-A) F, 5178 (C-A) F, 663 (A-G) F, 10398 (A-G) F, 10400 (G-A) R, 3594 (G-A) R, 7028 (G-A) R, 12406 (G-A) F, 4833 (A-G) F, and 7600 (G-A) F (FIG. 3). The primers designed in the reverse orientation incorporate the reverse complement nucleotide; therefore, the complementary base pair of the nucleotide incorporated during the reaction represents the nucleotide compared to the Cambridge reference sequence (CRS). As a result of the variable polymeric-T tails, the primers exhibited sufficient separation during electrophoresis to allow for simple identification and interpretation of the peaks. The ideal genomic DNA concentration range for the initial PCR reaction is between 0.2 and 0.5 ng/μL. Sensitivity tests indicated though that sufficient signal strength (300 relative fluorescent units and greater) could be obtained down to 0.007 ng of input genomic DNA. Adding more than 1 ng of genomic DNA to the reaction frequently resulted in off-scale peaks; however these generally did not compromise data interpretation. In the few cases in which the signal strength was high enough to complicate analysis, a reduced injection time was sufficient for successful troubleshooting. The 20 samples with known ancestral origin and haplogroup designation revealed consistent results, with a single exception. One sample previously typed as haplogroup D (5178A) using the RFLP system exhibited an additional mutation (1719A) found among individuals belonging to haplogroup X and I (data not shown). Of the 73 AFDIL samples for which a haplogroup was inferred based on CR sequence data, 59 samples gave identical results with the SNP typing. Of the 14 samples that did not give identical results, 3 samples were SNP typed as the same macrohaplogroup rather than the more specific haplogroup assignment based on the CR data, 5 samples gave an inconclusive haplogroup based on the SNP typing, and 6 samples were SNP typed as a different haplogroup than that determined from the CR data. Of the samples that gave an inconclusive haplogroup based on the SNP typing, 2 samples were SNP typed as the correct haplogroup but also had the 9 bp deletion found among haplogroup B individuals, 1 sample was heteroplasmic at position 10398, and 2 samples displayed mutations at both np 4833 and np 7600. Of the 6 samples that were SNP typed as a different haplogroup than that inferred from the CR data, 3 samples were SNP typed as haplogroup X or I based on a mutation at np 1719, 1 sample was SNP typed as L3 rather than the CR typed haplogroup D, and samples were SNP typed as haplogroup H rather than the CR typed haplogroup U. These results are summarized in Table 4. All 21 AFDIL samples for which a single haplogroup could not be inferred from CR sequence data were successfully SNP typed. Of these, one sample is known to have been incorrectly SNP typed as haplogroup I. The haplogroup I SNP type was based on the presence of the 1719A mutation, however the CR sequence is missing numerous mutations characteristic of haplogroup I (A16129G, C16223T, G16391A, T199C, T204C, T250C) (51). The results of the SNP typing of these 21 samples, including inconclusive haplogroup assignments (where available) based on the CR data, are summarized in Table 5. The samples of known ancestral origin but which had not been previously haplogroup typed were successfully SNP typed. These results are summarized in Table 6. The ancient skeletal remains also produced successful SNP typing results: samples 1 and 2, both extracted from World War II-era bones, SNP typed as haplogroup B (FIG. 4) and macrohaplogroup N (data not shown), respectively. All 12 SNPs tested were clearly typed in most of the tested samples, however all haplogroup A samples and five of the AFDIL haplogroup D samples exhibited drop out (null allele) of the peak corresponding to np 4833 and np 10398, respectively. A null allele can be caused by a failure of the initial amplification of the fragment encompassing the polymorphism or by a failed single base primer extension reaction. The null allele in the haplogroup A samples was most likely caused by an A to G transition (np 4824) in the 4833 extension primer annealing region (30), however all samples had the expected base substi-tutions 663A and 7028T. The five AFDIL samples that exhibited drop out of the 10398 SNP originated from the same region (Hong Kong), and are the only haplogroup D samples that exhibited this null allele. Coding region sequencing could be performed to determine the cause of the null allele, but such sequence data was not generated for these samples.

Discussion

The assay characterizes 12 mtDNA coding region polymorphisms selected to identify the European (haplogroup H and I), African (haplogroup L1/L2), and Asian/Native American (haplogroups E, F, G, A, B, C, and D) lineages. The assay is also able to identify macrohaplogroup L3, which includes several haplogroups in and out of Africa (52). When the SNP assay was applied to the 73 AFDIL samples previously haplogroup typed using CR sequence data, the correct haplogroup or macrohaplogroup was unambiguously identified ˜85% of the time. Among the 94 AFDIL samples SNP typed, a presumably correct haplogroup or macrohaplogroup was unambiguously assigned 87% of the time. As a first step screening tool for haplogroup assignment, the SNP typing compares quite favorably to the considerably more time consuming, expensive, and labor-intensive alternative method of CR sequencing that correctly inferred a specific haplogroup slightly less frequently. Of the AFDIL samples that did not result in a conclusive SNP haplogroup (5 samples, or ˜7%), four instances resulted from one of two mutations (7600A or the 9 bp deletion) identified in a sample of a different haplogroup, and one instance occurred as the result of heteroplasmy at np 10398. However, in each of these cases, the correct haplogroup was one of the two assigned. The 9 pb deletion that defines haplogroup B has occasionally been found in populations from different geographic origins, suggesting that this mutation has independently occurred more than once during human evolution (29,53,54). This explains why, in a small number of samples, the 9 bp deletion could be observed concurrent with a mutation specific for a different haplogroup. Overall, the data here demonstrate that inconclusive haplogroup assignment could be expected to occur less than 10% of the time. In practice, these few instances of inconclusive haplogroup assignment could be corrected by sequencing the CR or the coding region in the area of another haplogroup specific polymorphic site. Among the 7 AFDIL samples for which the SNP haplogroup disagreed with the CR-inferred haplogroup, more than half of the disagreements (4 samples) were the result of a mutation at np 1719. Thus, one limitation of this SNP assay is the correct identification of haplogroups X and I, as the mutation 1719A, initially chosen because found in both haplogroups, has also been observed in other haplogroups (10,26,30,38). It has been suggested that np 1719A is rather mu-table, thus accounting for the appearance of the base substitution in several geographic lineages (55). Consequently, in order to specifically identify haplogroup X, the 1719 SNP should be replaced with a more informative polymorphism (eg, 6371) that can distinguish haplogroup X from the root of macrohaplogroup N (29,33,55). Of the remaining 3 AFDIL samples for which the SNP and CR haplogroups disagreed, it is difficult to determine the correct haplogroup from the data available. In one case, the sample was tentatively designated as haplogroup D based on a specific CR mutation (16362C), while the SNP typing resulted in a L3 assignment. Like np 1719, the 16362 mutation has been observed on multiple branches of the mtDNA tree and may mutate frequently (32-34). As such, the CR haplogroup assignment may be incorrect for this sample. In the second and third case, the samples were designated as haplogroup U based on a specific mutation in the CR, but the SNP typing identified these samples as haplogroup H due to the lack of mutation at np 7028. Sequencing relevant portions of the coding region, or SNP typing of additional haplogroup specific positions of interest, would be the best (and likely only) way to resolve these haplogroup disagreements. One area in which the SNP haplogroup typing proved to have a significant advantage over the CR haplogroup typing was in the capability to distinguish between haplogroup D and haplogroup G. More than half of the samples for which a single haplogroup could not be inferred on the basis of the CR sequence data were believed to belong to either haplogroup D or haplogroup G. In each of these cases, the SNP typing confidently assigned the samples to one of these two haplogroups (Table 5). World War II-era Sample 1 was a human femur whose potential origin was an American soldier killed in 1945 in a plane crash in the Philippine Islands. In late 1946, American personnel visited the crash site and recovered wreckage but no human remains. In July of 1950, a Filipino native contacted the US Army and provided the femur, identifying the crash site as its source. However, in order to identify the femur as that of the missing American soldier, the mtDNA data would need to be consistent with the H haplogroup of the soldier's maternal relatives. The SNP typing of Sample 1 identified the sample as haplogroup B, thus excluding the American soldier as the source of the sample. Sample 2 was a human femur found in the wreckage of a B-25D-1 Mitchell bomber that crashed in New Guinea in 1943 after being attacked by Japanese aircraft. Human remains from the crash were initially recovered and buried in New Guinea in the late 1940s, and later exhumed in 1947 for transfer to the Philippine Islands for storage. Before re-interment in 1950 at the Manila American Military Cemetery, the remains were treated with preservatives, and then finally exhumed again in 2004 for identification purposes. Four American soldiers were aboard the B25D-1 Mitchell bomber at the time of the crash. The mtDNA haplogroups of each were determined using maternal references: 2 soldiers belonged to haplogroup H, the third soldier to haplogroup T, and the last soldier to haplogroup K. SNP typing of the human femur submitted to AFDIL for analysis identified the sample as haplogroup N, thus excluding the two haplogroup H soldiers. In these two cases, the SNP typing of the ancient skeletal remains confirmed the haplogroup obtained by CR sequencing. Though amplification and sequencing of the CR was successful when amplicons smaller than 150 bp were targeted, obtaining CR sequence data are generally time consuming, expensive, labor intensive, and can consume large quantities of limited extract. In both cases, the SNP typing confirmed the haplogroups identified by CR sequence data and resolved questions relating to the source of the femurs with a quick and easy assay that required only a single multiplexed amplification. Based upon the specific SNPs included in this assay, only limited information was obtained from certain samples. For example, 7028T excludes a sample from haplogroup H, however the sample can still be placed within macrohaplogroup N. This macrohaplogroup contains mtDNA types from the European, Asian, and Native American lineages, thus providing inconclusive information on ancestral origin. The inconclusive ancestral determination is currently being addressed in our laboratories with the development of a European haplogroup assay (using the same minisequencing technology) designed to simultaneously type additional SNPs that allow identification of haplogroups H, H1, I, J, K, T, U, V, W, and X. The multiplex assay is currently being tested in our laboratories. In conclusion, the minisequencing method utilized for this panel of SNPs demonstrated the ability to rapidly screen for haplogroups A, B, C, D, E, F, G, H, L1/L2, L3, M, and N when working either pristine or highly degraded DNA samples, therefore making it a potentially useful screening tool for molecular anthropology studies. Furthermore, the minisequencing strategy allowed for simple multiplexing, the design of amplicons with minimal length, and the ability to target multiple nucleotide positions located throughout the mtDNA coding region. These properties reduce sample consumption and enable simplistic interpretation of multiple SNPs simultaneously, which are desirable qualities for forensic DNA analysis.

Determination of European Specific Haplogroups

The purpose of this part of the study was to develop a single multiplex that could characterize sixteen polymorphic sites that correspond to the nine major European haplogroups. A single multiplex uses up less sample than if multiple amplifications are needed and the desire for the development of a single multiplex using the single base primer extension method for coding region mtDNA SNPs is due to the need for a method that can quickly and accurately characterize degraded samples, both for forensic human identification interests and phylogenetic interests. The European haplogroups were targeted because a global haplogroup typing kit for the major African, Asian, Native American, and European haplogroups had previously been developed by Tahnee Nelson, MSFS at The George Washington University (see above); her kit contained the haplogroups H, I and X, but did not target any of the other European haplogroups. This European haplogroup kit is intended to for use in conjunction with Ms. Nelson's global haplogroup kit to allow for a method to quickly screen degraded samples and narrow down the number of reference samples that would need to be sequenced, thus saving time, labor, and resources. The single base primer extension method was chosen because of its ability to multiplex and its adaptability to the fluorescent fragment detection platforms, such as analysis with the ABI Genetic Analyzer 3130, that are prevalent in forensic laboratories.

Selection of the SNP's—European Haplogroups

The single nucleotide polymorphisms for this multiplex assay were chosen based on the SNPs defining the nine major European haplogroups as listed in Brandstätter et al. (2003) and supplemented with other European haplogroup-specific SNPs described in other literature. These SNPs were chosen so that many were specific to one of the nine major haplogroups plus H1, a sub-haplogroup of H. Haplogroup H is the largest European haplogroup, thus sub-haplogroup H1, the largest within H, was chosen for analysis to provide further discrimination. European continent-specific polymorphisms were chosen as a continuation of the global kit.

Haplogroup H is separated from the other non-H haplogroups by exhibiting a C at the C to T polymorphism at nucleotide position 7028 (C7028T); all non-H European haplogroups possess a T at that site, whereas all the sub-haplogroups of H retain the C at position 7028. Haplogroups H and V are then separated from the rest of the haplogroups by displaying a C at the C to T polymorphism at position 14766 (C14766T). This is in agreement with phylogenetic trees (FIG. 1.9) that have shown haplogroups H and V as belonging to a single cluster, HV.

Haplogroups J and T share the A to G polymorphism at position 11251 (A11251G) and the T to C polymorphism at position 4216 (T4216C). This is also in agreement with the phylogenetic tree (FIG. 1.9) that presents haplogroups J and T as belonging to the same over-arching haplogroup JT. However, haplogroup J is individuated by the G to A polymorphism at position 13708 (G13708A), whereas haplogroup T is differentiated by two different polymorphisms: G to A at position 709 (G709A) and G to A at position 8697 (G8697A). The latter is specific for haplogroup T, however G709A is shared with haplogroup W; this is in accordance with the parsimony tree (FIG. 1.10) displaying groups T and W as have more in common than haplogroups J and T. Haplogroups K and U also share two polymorphisms, which is not unexpected as several sources have reported that K diverged directly from haplogroup U. The shared polymorphisms are an A at the G to A polymorphism at position 12372 (G12372A) and a G at the A to G polymorphism at position 12308 (A12308G). Haplogroup U is defined by these two polymorphisms, in addition to the C7028T shared with all other non-H haplogroups and the C14766T shared with the non-HV haplogroups. Haplogroup T is differentiated from haplogroup U with the addition of an A to G polymorphism at position 1811 (A1811 G) and a T to C polymorphism at position 14798 (T14798C), both of which are specific for haplogroup K. Furthermore, haplogroup K shares the G to A polymorphism at position 9055 (G9055A) with haplogroup I. Although a phylogeny tree demonstrates no direct link between the two haplogroups (FIG. 1.8), a parsimony analysis shows haplogroups I and K residing on the same branch (FIG. 1.9).

Haplogroups I and W share the polymorphism G to A at position 8251 (G8251), in addition to the more broadly shared C7028T and C14766T polymorphisms. For further discrimination, haplogroup W displays the G709A polymorphism and haplogroup I exhibits the G9055A. Although these polymorphisms are shared with haplogroups T and K, respectively, they clearly represent their respective haplogroups when present in conjunction with the G9055A, C7028T, and C14766T polymorphisms. Haplogroup X, having descended directly from the macro-haplogroup N, displays only the C7028T and C14766T polymorphisms. Identification of a sample as belonging to haplogroup X is more of a vague categorization than a true identification due to the fact that haplogroup X is an underived haplogroup and shares its characteristic SNPs with many other haplogroups.

When viewed together, these polymorphisms may be arranged in a diagram that will aid in haplogroup assignment. FIG. 2.1 mostly indicates the non-CRS base, labeled in red, that is the basis of the defining polymorphism; however, the CRS base, if necessary to include on the diagram, is labeled in black. This designation tree assumes that all other SNPs correspond with the CRS unless a polymorphism has been noted, with the polymorphisms accumulating up to the designated haplogroup.

Primer Design—European Haplogroups

PCR primers were designed with the aid of the PrimerQuestSM software offered by Integrated DNA Technologies (IDT) on their website. (http://www.idtdna.com/Scitools/Applications/Primerquest/) The sequence of a large section of mtDNA containing the desired region is inputted to the program, having specified the range of parameters, including length of amplicon, desired annealing temperature, and desired GC content. The program then returns several options for primers that correspond to the requested criteria. The primer amplicon lengths ranged from 60 base pairs to 130 base pairs and primer length was between 22 and 26 base pairs. The targeted annealing temperature was between 55° and 60° C., while the targeted GC content was 50%; the most important feature of the temperature was that, more than being between 55° and 60° C., the annealing temperature of both primers in a set needed to be as close as possible or ideally identical.

Due to the desired application of this multiplex kit for degraded DNA samples, several criteria were considered when designing the PCR primers. These criteria were that: the PCR primers must flank the SNP site, the amplicon size must not exceed 130 base pairs, and the amplicon must retain the minisequencing primer annealing site in order to ensure accurate single base extension. (Nelson, 2006) An amplicon size around or under 100 base pairs would have been ideal; however, SNPs A12308G and G12372A were too close to each be on their own amplicon, separated by only 64 bases, and satisfy the criteria of retaining the minisequencing primer annealing site, in addition to being able to adhere to the desired primer parameters. Furthermore, the PCR primers that would produce amplicons for each of these SNPs would most likely overlap and would be therefore be prone to primer-dimer formation; consequently, the amplicon length is 130 base pairs, instead of the ideally smaller amplicon size. This was also the case for SNPs C14766T and T14798C, which are separated by only 32 bases; however, because these SNPs were closer together than the 12308/12372 polymorphisms, the amplicon length was able to be kept at 110 bases. The minisequencing primers were designed using the same procedure as for the “global” haplogroup primers: Primer Express Version 2.0 was used for the design and were added to differentiate electrophoretic mobility and facilitate SNP typing.

PCR Amplification

The amplification procedure was carried out in a 50 μL reaction, containing 0.035 units of AmpliTaq Gold® DNA Polymerase (Applied Biosystems, Foster City, Calif.), 1× GeneAmp® PCR Gold Buffer (Applied Biosystems, Foster City, Calif.), 20 mM MgCl2 (Applied Biosystems, Foster City, Calif.), 200 μM of each dNTP (Roche, Mannheim, Germany), 1 μL of DNA extract, and 7.5 μL of sterile DNA-grade dH2O (Fisher Scientific, Fair Lawn, N.J.). Final primer concentrations for each PCR reaction: 709 primer set at 0.04 μM, the 11251 primer set at 0.12 μM, the 7025 and 15904 primer sets at 0.16 μM, the 8697 primer set at 0.18 μM, the 3010, 9055, and 13708 primer sets at 0.2 μM, the 4216 primer set at 0.22 μM, and the 8251 primer set at 0.3 μM. 14766/14798 primer set at 0.4 μM, the 1811 and 12308/12372 primer sets at 0.6 μM, and the 4580 primer set at 0.8 μM.

PCR was carried out using the same amplification protocol used for the global assay on a GeneAmp® PCR System 9600 thermalcycler (Applied Biosystems, Foster City, Calif.) and purification of PCR product was performed using Exonuclease I (Exo) (USB, Cleveland, Ohio) and Shrimp Alkaline Phosphatase (SAP) (Roche Diagnostics Corporation, Indianapolis, Ind.) as previously described.

Minisequencing

The 16 primer minisequencing multiplex reaction was performed using, 3 μL of SNaPshot™ Multiplex Ready Reaction Mix (Applied Biosystems, Foster City, Calif.) and 2 microliter of purified PCR product. Water and minisequencing primer mix was added to a final volume of 10 microliters. The final minisequencing primer concentrations were: 0.033 μM for the SNPs at position 12372, 0.050 μM for SNPs at positions 709, 1811, and 4216, 0.083 μM for SNPs at positions 3010, 7028, 8697, 9055, 11251, and 15904, and 0.124 μM for the polymorphism at position 8251; 0.221 μM for SNPs at positions 13708, 14766, and 14798, 0.276 μM for the polymorphism at position 12308, and 0.552 μM for the SNP located at position 4580.

The minisequencing reaction and SAP purification were performed using the same thermocycling procedure as the global assay.

Preparation and Analysis of MS Product onto ABI 3130

2 μL of the purified minisequencing product is added to 10 μL of a highly deionized formamide (HIDI) and Liz-120 Size Standard (Applied Biosystems, Foster City, Calif.) mix. Samples were loaded on an ABI 3130 Genetic Analyzer following the procedure previously described and ran using pop7: parameters were set as follows: 6 sec. injection time and 1.2 kV injection voltage. E5dye set 60° C. over temp., 15.0 kV run voltage and 600 sec. of run time.

Results—European Haplogroup

3.1—Multiplexed Single Nucleotide Polymorphism Sites

As previously discussed, there are several single nucleotide polymorphisms (SNPs) that are characteristic of the nine major European haplogroups: H, I, J, K, T, U, V, W, and X, in addition to the H1 sub-haplogroup of haplogroup H. The final order of SNP migration is as follows: A1811G, G12372A, G13708A, A12308G, G709A, G3010A, G8251A, G9055A, C7028T, C15904T, A11251G, T4216C, G4580A, G8697A, T14798C, and C14766T. Primers for G709A and G3010A, as well as G9055A and C7028T, overlap but this is not problematic because one out of the two SNPs was designed to display a C to T and the other was designed to display G to A. Haplogroup H is characterized by a G (blue peak) for marker 7028, which corresponds to the presence of a C on the forward strand. Haplogroup H1 is exhibits a T (red peak) at marker 3010, corresponding to an A on the forward strand, in addition to the G (blue peak) at 7028. Haplogroup V is characterized by an A (green peak) instead of a G at marker 7028 in addition to the presence of a T (red peak) at markers 15904 and 4580. All subsequent non-H haplogroups will display an A (green peak) at marker 7028 and all haplogroups that are neither H nor V will display a C (black peak) at marker 14766, instead of the T (red peak) present at that marker for haplogroups H, H1, and V.

Haplogroup J will display a C (black peak) at marker 11251 and a G (blue peak) at marker 4216, which are also present in haplogroup T. To individuate haplogroup J, a T (red peak) is present at marker 13708. An A (green peak) at marker 709 and a T (red peak) at marker 8697 characterize haplogroup T. A sample was analyzed that was determined to be haplogroup J; however, a T (red peak) was present at position 3010 instead of the typical C (black peak). Although 3010T characterizes haplogroup H1, a variant of haplogroup J containing 3010T has been seen before, for instance in Brandstatter et al. (2003)

Haplogroups U and K both have an A (green peak) at marker 12372, as well as a C (black peak) at marker 12308. These two peaks are characteristic of haplogroup U; however, haplogroup K is identified by the presence of an A (green peak) at marker 1811, a C (black peak) at 9055, and an A (green peak) at marker 14798.

Haplogroup X is represented only by an A (green peak) at marker 7028 and a T (red peak) at marker 14766. Haplogroup W is characterized by an additional two SNPs, the presence of an A (green peak) at marker 709 and the presence of a T (red peak) at marker 8251. Unfortunately, haplogroup I is one of the rarest European haplogroups and was not encountered in testing randomly chosen Caucasian samples. However, a T (red peak) at markers 8251 and 9055 would be expected.

The samples were screened using a previous version of the multiplex and samples were chosen that possess polymorphisms at each of the SNP sites. Each sample exhibited a different set of polymorphisms and, as expected, represented a different haplogroup. Two samples were chosen that were thought to represent haplogroup H, due to the lack of other polymorphisms, and the two samples were confirmed as haplogroup H.

Other Caucasian samples were later tested and the data was added to the previous samples, for a total of 20 Caucasian samples. Five samples of Asian origin and eight African samples were also tested; the data is represented in Table 3.2. Other variants of haplogroups X, U, and K were identified for the Caucasian samples, whereas the Asian and African samples contained ambiguous sets of polymorphisms that resulted in non-identification of a haplogroup. Many of the Asian and African samples were identified as X, but as previously explained, identification of haplogroup X is not so much identification as it is a vague categorization. Only the Caucasian samples were able to be accurately typed, whereas the typing for the Asian and African samples was mostly ambiguous or unknown. All haplogroup variants, except for haplogroup X, in Caucasian samples have previously been seen in Brandstatter et al. 2003.

Discussion

A multiplex has been developed for the characterization of the nine European haplogroups, H, I, J, K, T, U, V, W, and X, in addition to the sub-haplogroup H1 using polymorphisms found in the coding region of the mitochondrial DNA (mtDNA) genome. The multiplex successfully utilized the single base primer extension, or minisequencing, method to characterize the single nucleotide polymorphisms (SNPs) that are indicative of European haplogroups with a specific combination of polymorphisms; short amplicons less than 150 base pairs are more successful in detecting degraded mtDNA and the minisequencing method effectively detected the shortened amplicons. Several samples were characterized and exhibited the core polymorphisms of eight out of nine major European haplogroups and sub-haplogroup H1. Haplogroup I was not observed most likely because it is one of the least frequent haplogroups in European populations and the sample size was too small. It is important to note that the origin of a person's mitochondrial DNA does not speak to their physical appearance and should not be used to identify those features. The use of continent-specific haplogroups in forensic human identification is to provide a genetic method of identification that happens to be associated with a specific origin; the use of the minisequencing method in conjunction with mtDNA SNPs is a quick, effective, and cost-efficient method for mtDNA analysis when compared to traditional mtDNA sequencing methods.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

TABLE 1

| | Nucleotide position and base | | | | | | | | | | | | |
|---|---|---|---|---|---|---|---|---|---|---|---|---|---|
| | 8272- | | | | | | | | | | | | |
| Haplogroup | 8280 del | 13263 | 1719 | 5178 | 663 | 10398 | 10400 ^{r} | 3594 ^{r} | 7028 ^{r} | 12406 | 4833 | 7600 | Inferred ancestry |
| A | C | A | G | C | B | A | C | C | T | G | A | G | Asian/Native American |
| B | G | A | G | C | A | A | C | C | T | G | A | G | Asian/Native American |
| C | C | G | G | C | A | G | T | C | T | G | A | G | Asian/Native American |
| D | C | A | G | A | A | G | T | C | T | G | A | G | Asian/Native American |
| E | C | A | G | C | A | G | T | C | T | G | A | A | Asian |
| F | C | A | G | C | A | A | C | C | T | A | A | G | Asian |
| G | C | A | G | C | A | G | T | C | T | G | G | G | Asian |
| H | C | A | G | C | A | A | C | C | C | G | A | G | European |
| I | C | A | A | C | A | G | C | C | T | G | A | G | European |
| L1/L2 | C | A | G | C | A | G | C | T | T | G | A | G | African |
| L3 | C | A | G | C | A | G | C | C | T | G | A | G | African/European/Asian |
| M | C | A | G | C | A | G | T | C | T | G | A | G | Asian/Native American |
| N | C | A | G | C | A | A | C | C | T | G | A | G | European/Asian/Native |
| | | | | | | | | | | | | | American |
| X | C | A | A | C | A | A | C | C | T | G | A | G | European/Asian/Native |
| | | | | | | | | | | | | | American |

*Abbreviation: ^{r} - the single base extension primer is in the reverse orientation.

TABLE 2

| | | | | | | | |
|---|---|---|---|---|---|---|---|
| | | | Primer | | | GC | |
| Nucleotide | | | length | Amplicon | Tm | content | Final |
| position | Primer sequence (5′→3′) | Orientation | (bp) | length (bp) | (° C.) | (%) | (nM) |

| | | | | | | | |
|---|---|---|---|---|---|---|---|
| 8272-8280 del | TAAAAATCTTTGAAATAGGGCCC | F | 23 | 89 (del) | 51.4 | 34.7 | 200 |
| | GTTAATGCTAAGTTAGCTTTACAGTGG | R | 27 | 80 | 54.2 | 37 | |
| 13263 | CAAAAAAATCGTAGCCTTCTCC | F | 22 | 67 | 52.2 | 40.9 | 150 |
| | GTTGATGCCGATTGTAACTATTATG | R | 25 | | 52.3 | 36 | |
| 1719 | CCCACTCCACCTTACTACCAGA | F | 22 | 84 | 57.7 | 54.5 | 500 |
| | TGCGCCAGGTTTCAATTT | R | 18 | | 53.1 | 44.4 | |
| 5178 | TAAACTCCAGCACCACGACC | F | 20 | 79 | 57.2 | 55 | 200 |
| | GTGGATGGAATTAAGGGTGTTAG | R | 23 | | 53.3 | 43.4 | |
| 663 | ACATCACCCCATAAACAAATAGG | F | 23 | 108 | 52.9 | 39.1 | 200 |
| | TGGTGATTTAGAGGGTGAACTCA | R | 23 | | 55.6 | 43.4 | |
| 10398/10400 | AGTCTGGCCTATGAGTGACTAC | F | 22 | 86 | 55.5 | 50 | 500 |
| | AATGAGTCGAAATCATTCGTTT | R | 22 | | 50.7 | 31.8 | |
| 3594 | CTTAGCTCTCACCATCGCTCT | F | 21 | 90 | 56.2 | 52.3 | 300 |
| | AGAATAAATAGGAGGCCTAGGTTG | R | 24 | | 53.9 | 41.6 | |
| 7028 | TATTAGCAAACTCATCACTAGACA TCGT | F | 28 | 96 | 55.7 | 35.7 | 200 |
| | TGGCAAATACAGCTCCTATTGA | R | 22 | | 54 | 40.9 | |
| 12406 | AATTCCCCCCATCCTTACC | F | 19 | 78 | 54.2 | 52.6 | 300 |
| | GCGACAATGGATTTTACATAATG | R | 23 | | 50.6 | 34.7 | |
| 4833 | AATAGCCCCCTTTCACTTCTG | F | 21 | 72 | 54.7 | 47.6 | 400 |
| | AGAAGAAGCAGGCCGGA | R | 17 | | 56.4 | 58.8 | |
| 7600 | GGCTAAATCCTATATATCTTAATGGCA | F | 27 | 64 | 52.6 | 33.3 | 100 |
| | GGGAAGTAGCGTCTTGTAGACC | R | 22 | | 59.9 | 54.5 | |

TABLE 2.1

| | | | | | |
|---|---|---|---|---|---|
| | | Primer Length | Amplicon Length | | |
| Locus (np), F/R | Primer Sequence | (bp) | (bp) | Tm (° C.) | GC (%) |

| | | | | | |
|---|---|---|---|---|---|
| mt709 F | TGGTCCTAGCCTTTCTATTAGCTC | 24 | 85 | 55.5° | 45.8% |
| mt709 R | CGTGGTGATTTAGAGGGTGAACTC | 24 | | 57.0° | 50.0% |
| mt1811 F | GCGATAGAAATTGAAACCTGGCG | 23 | 92 | 56.5° | 47.8% |
| mt1811 R | GGGTTAGTCCTTGCTATATTATGCT | 25 | | 54.1° | 40.0% |
| mt3010 F | ACGACCTCGATGTTGGATCAGGA | 23 | 98 | 59.5° | 52.2% |
| mt3O10 R | GGTCTGAACTCAGATCACGTAGGACT | 26 | | 59.0° | 50.0% |
| mt4216 F | TTCCGCTACGACCAACTCATACAC | 24 | 108 | 58.4° | 50.0% |
| mt4216 R | GGGAATGCTGGAGATTGTAATGGG | 24 | | 57.6° | 50.0% |
| mt4580 F | ACAGCGCTAAGCTCGCACTGATTT | 24 | 116 | 60.9° | 50.0% |
| mt4580 R | TTGATGGCAGCTTCTGTGGAACGA | 24 | | 60.6° | 50.0% |
| mt7028 F | TATTAGCAAACTCATCACTAGACATCGT | 28 | 96 | 55.7° | 35.7% |
| mt7028 R | TGGCAAATACAGCTCCTATTGA | 22 | | 54.0° | 40.9% |
| mt8251 F | AACCACAGTTTCATGCCCATCGTC | 24 | 122 | 59.6° | 50.0% |
| mt8251 R | CTAAGTTAGCTTTACAGTGGGCTCT | 25 | | 56.0° | 44.0% |
| mt8697 F | CAACAACCGACTAATCACCACCCA | 24 | 90 | 58.9° | 50.0% |
| mt8697 R | CAGGTTCGTCCTTTAGTGTTGTGT | 24 | | 56.9° | 45.8% |
| mt9055 F | GCCACCTACTCATGCACCTAATTG | 24 | 70 | 57.6° | 50.0% |
| mt9055 R | AGTGTAGAGGGAAGGTTAATGGTTG | 25 | | 56.1° | 44.0% |
| mt11251 F | CCTGAACGCAGGCACATACTTCCTAT | 26 | 94 | 60.2° | 50.0% |
| mt11251 R | TGAGCCTAGGGTGTTGTGAGTGTA | 24 | | 59.1° | 50.0% |
| mt12308/12372 F | CAGCTATCCATTGGTCTTAGGC | 22 | 130 | 55.0° | 50.0% |
| mt12308/12372 R | TTAACGAGGGTGGTAAGGATGG | 22 | | 56.0° | 50.0% |
| mt13708 F | ACCCTAACAGGTCAACCTCGCTTC | 24 | 113 | 60.4° | 54.2% |
| MT13708 R | AGAAATCCTGCGAATAGGCTTCCG | 24 | | 58.8° | 50.0% |
| mt14766/14798 F | CAACTACAAGAACACCAATGACCC | 24 | 110 | 55.8° | 45.8% |
| MT14766/14798 R | TCATCATGCGOAGATGTTGGATGG | 24 | | 58.9° | 50.0% |
| mt15904 F | ACTCAAATGGGCCTGTCCTTGTAG | 24 | 60 | 58.7° | 50.0% |
| mt15904 R | CATCTCCGGTTTACAAGACTGGTGT | 25 | | 58.4° | 48.0% |

TABLE 2.2

| Locus (np), base | Observed | | Final Primer | | |
|---|---|---|---|---|---|
| change, F/R | Base Change | Sequence | Length | % GC | Tm |
| mt13708g/a R | C/T | GAATAGGCTTCCGGCTG | 17 | 59 | 53 |
| mT1811 a/g F | A/G | GCAAGGGAAAGATGAAAAATTATA | 27 | 29 | 55 |
| mt12372 g/a F | G/A | (4T)-ACACTACTATAACCACCCTAACCCT | 29 | 44 | 55 |
| mt3010 g/a R | C/T | (11T)-TTAATAGCGGCTGCACCAT | 30 | 47 | 56 |
| mt12308 a/g R | T/C | (13T)-TTGGAGTTGCACCAAAATT | 32 | 37 | 53 |
| mt1709 g/a F | G/A | (22T)-TACACATGCAAGCATCCCC | 41 | 47 | 54 |
| mt8251 g/a R | C/T | (20T)-GGTGCTATAGGGTAAATACGGG | 42 | 50 | 56 |
| mt9055 g/a R | C/T | (27T)-TGGTTGATATTGCTAGGGTGG | 48 | 48 | 56 |
| mt15904 c/t F | C/T | (31T)-GGGCCTGTCCTTGTAGTATAAA | 53 | 45 | 54 |
| mt11251 a/g R | T/C | (35T)-CCTAGGGTGTTGTGAGTGTAAAT | 58 | 43 | 54 |
| mt4216 t/c R | A/G | (38T)-GAGATTGTAATGGGTATGGAGACAT | 63 | 40 | 56 |
| mt14580 g/a R | C/T | (42T)-TTGGTTAGAACTGGAATAAAAGCTAG | 68 | 35 | 56 |
| mt8697 g/a R | C/T | (47T)-CGTCCTTTAGTGTTGTGTATGGTTAT | 73 | 38 | 57 |
| mt14798 t/c R | A/G | (61T)-GGTGGGGAGGTCGATGA | 78 | 65 | 56 |
| mt14766 c/t F | C/T | (64T)-ATGACCCCAATACGCAAAA | 83 | 42 | 55 |

TABLE 3

| | | | Primer | | GC | | Base |
|---|---|---|---|---|---|---|---|
| Nucleotide | Primer sequence | Orien- | length | | content | Final | substi- |
| position | (5′→3′) | tation | (bp) | Tm (° C.) | (%) | (nM) | tution |

| | | | | | | | |
|---|---|---|---|---|---|---|---|
| 8272-8280 del | CCCTATAGCACCCCCTCTA | F | 19 | 54.9 | 57.8 | 84 | C > G |
| 13263 | (3-poly-T tail)-TAGCCTTCTCCACTTCAAGTCA | F | 25 | 56.3 | 40.0 | 83 | A > G |
| 1719 | (9-poly-T tail)-CACTCCACCTTACTACCAGACAAC | F | 33 | 59.2 | 36.3 | 292 | G > A |
| 5178 | (13-poly-T tail)-CTACTATCTCGCACCTGAAACAAG | F | 37 | 58.5 | 29.7 | 167 | C > A |
| 663 | (19-poly-T tail)-CCATAAACAAATAGGTTTGGTCCT | F | 43 | 58.8 | 20.9 | 84 | A > G |
| 10398 | (21-poly-T tail)- | F | 48 | 59.7 | 20.8 | 292 | A > G |
| | GAGTGACTACAAAAAGGATTAGACTGA | | | | | | |
| 10400 | (24-poly-T tail)- | R | 53 | 58.4 | 13.2 | 292 | C > T |
| | TTCGTTTTGTTTAAACTATATACCAATTC | | | | | | |
| 3594 | (29-poly-T tail)-TAGGAGGCCTAGGTTGAGGTT | R | 58 | 62.2 | 20.6 | 167 | C > T |
| 7028 | (33-poly-T tail)- | R | 63 | 62.6 | 20.6 | 84 | C > T |
| | CCTATTGATAGGACATAGTGGAAGTG | | | | | | |
| 12406 | (50-poly-T tail)-CCCATCCTTACCACCCTC | F | 68 | 63.5 | 16.1 | 167 | G > A |
| 4833 | (54-poly-T tail)-CCAGAGGTTACCCAAGGC | F | 73 | 64.7 | 16.4 | 292 | A > G |
| 7600 | (51-poly-T tail)-TATCTTAATGGCACATGCAGC | F | 78 | 64.1 | 12.8 | 166 | G > A |

TABLE 4

| No. of samples (n = 73) | CR haplogroup | SNP haplogroup |
|---|---|---|
| 9 | A | A |
| 4 | B | B |
| 5 | C | C |
| 1 | C | M* |
| 7 | D | D |
| 1 | D | L3^{‡} |
| 1 | F | F |
| 2 | F | N* |
| 2 | G | E or G^{†} |
| 3 | H | H |
| 1 | HV | N |
| 1 | I | I |
| 1 | I | I or X^{†} |
| 1 | I | I + 9 bp deletion^{†} |
| 5 | L | L |
| 1 | L | L + 9 bp deletion^{†} |
| 11 | L3 | L3 |
| 1 | L3 | I^{‡} |
| 7 | M | M |
| 2 | N | X^{‡} |
| 3 | U | N |
| 2 | U | H^{‡} |
| 2 | X | X |

*SNP typing identified the correct macrohaplogroup but failed to determine the more specific haplogroup assigned based on the CR data (3 samples).
^{†}SNP typing did not result in a conclusive haplogroup (5 samples).
^{‡}CR and SNP haplogroup typing results did not agree (6 samples).

TABLE 5
Table 5. Single nucleotide polymorphisms (SNP) typing results
for Armed Forces DNA Identification assignment.

| No. of samples (n = 21) | CR haplogroup | SNP haplogroup |
|---|---|---|
| 10 | D or G | D |
| 1 | D or G | G |
| 1 | ? | H |
| 1 | ? | I* |
| 4 | ? | N |
| 4 | ? | L3 |

Twenty one: haplogroup determined by G″ haplogroups by CR
*One sample is known to have been incorrectly SNP typed.

TABLE 6

| Known ancestral | Haplogroup | |
|---|---|---|
| origin | designation | Inferred ancestry* |
| EU | N | EU/AS/NA |
| EU | I | EU |
| EU | H | EU |
| EU | N | EU/AS/NA |
| EU | H | EU |
| EU | N | EU/AS/NA |
| EU | N | EU/AS/NA |
| EU | L3 | EU/AF/AS |
| EU | B | AS/NA |
| EU | H | EU |
| EU | N | EU/AS/NA |
| AF | L1/L2 | AF |
| AF | L1/L2 | AF |
| AF | L1/L2 | AF |
| AF | L1/L2 | AF |
| AF | L3 | EU/AF/AS |
| AF | L1/L2 | AF |
| AF | N | EU/AS/NA |
| AF | N | EU/AS/NA |
| AF | L1/L2 | AF |
| AS | D | AS/NA |
| AS | N | EU/AS/NA |
| AS | D | AS/NA |
| AS | L3 | EU/AF/AS |
| AS | M | AS/NA |
| AS | B | AS/NA |
| AS | M | AS/NA |
| AS | N | EU/AS/NA |
| AS | L3 | EU/AF/AS |
| AS | F | AS |
| AS | N | EU/AS/NA |

*Abbreviations: EU—European; AF—African; AS—Asian; NA—Native American

What is claimed:

1. A SNP multiplex reaction, comprising: (i) obtaining a sample of human mitochondrial DNA; and (ii) screening the mitochondrial DNA using a PCR primer set and a minisequencing primer set, wherein the reaction identifies the nucleotide present at 12 locations within mtDNA, wherein the locations comprise haplogroups A, B, C, D, E, F, G, H, I, L1/L2, M, or N, wherein the sample is from a limited amount of starting material, and wherein maternal ancestry of the sample is rapidly inferred and the haplogroup of the human individual is assigned following a single reaction.

2. A method for identifying polymorphisms in a sample of human mitochondrial DNA, comprising: (i) obtaining the mitochondrial DNA from the sample; (ii) screening the mitochondrial DNA using a PCR primer set and a minisequencing primer set, wherein the primer sets are directed to haplogroups A, B, C, D, E, F, G, H, I, L, M, N, and X, said PCR primer set comprising primers described in Table 2 (SEQ ID NOS 1-22, respectively, in order of appearance) and said minisequencing primer set comprising primers described in Table 3 (SEQ ID NOS 23-34, respectively, in order of appearance).

3. A PCR primer set, comprising Table 2 (SEQ ID NOS 1-22, respectively, in order of appearance).

4. A minisequencing primer set, comprising Table 3 (SEQ ID NOS 23-34, respectively, in order of appearance).

5. A kit for performing the method of claim 1.

6. A method for identifying polymorphisms in a sample of human mitochondrial DNA, comprising: (i) obtaining the mitochondrial DNA from the sample; (ii) screening the mitochondrial DNA using a PCR primer set and a minisequencing primer set, wherein the primer sets are directed to 16 SNPs that include the diagnostic polymorphic sites for the European haplogroups H, J, K, T, U, V, and W, said PCR primer set comprising primers described in Table 2.1 (SEQ ID NOS 35-45, 16, 46-61, respectively, in order of appearance) and said minisequencing primer set comprising primers described in Table 2.2 (SEQ ID NOS 62-76, respectively, in order of appearance).

7. A PCR primer set, comprising: primers described in Table 2.1 (SEQ ID NOS 35-45, 16, 46-61, respectively, in order of appearance).

8. A minisequencing primer set, comprising: primers described in Table 2.2 (SEQ ID NOS 62-76, respectively, in order of appearance)

9. A kit for performing the method of claim 5.


SEQUENCE LISTING (76 sequences)

SEQ ID NO: 1 - DNA, 23 bp, Artificial Sequence
  Description of Artificial Sequence Synthetic primer
taaaaatctttgaaatagggccc

SEQ ID NO: 2 - DNA, 27 bp, Artificial Sequence
  Description of Artificial Sequence Synthetic primer
gttaatgctaagttagctttacagtgg

SEQ ID NO: 3 - DNA, 22 bp, Artificial Sequence
  Description of Artificial Sequence Synthetic primer
caaaaaaatcgtagccttctcc

SEQ ID NO: 4 - DNA, 25 bp, Artificial Sequence
  Description of Artificial Sequence Synthetic primer
gttgatgccgattgtaactattatg

SEQ ID NO: 5 - DNA, 22 bp, Artificial Sequence
  Description of Artificial Sequence Synthetic primer
cccactccaccttactaccaga

SEQ ID NO: 6 - DNA, 18 bp, Artificial Sequence
  Description of Artificial Sequence Synthetic primer
tgcgccaggtttcaattt

SEQ ID NO: 7 - DNA, 20 bp, Artificial Sequence
  Description of Artificial Sequence Synthetic primer
taaactccagcaccacgacc

SEQ ID NO: 8 - DNA, 23 bp, Artificial Sequence
  Description of Artificial Sequence Synthetic primer
gtggatggaattaagggtgttag

SEQ ID NO: 9 - DNA, 23 bp, Artificial Sequence
  Description of Artificial Sequence Synthetic primer
acatcaccccataaacaaatagg

SEQ ID NO: 10 - DNA, 23 bp, Artificial Sequence
  Description of Artificial Sequence Synthetic primer
tggtgatttagagggtgaactca

SEQ ID NO: 11 - DNA, 22 bp, Artificial Sequence
  Description of Artificial Sequence Synthetic primer
agtctggcctatgagtgactac

SEQ ID NO: 12 - DNA, 22 bp, Artificial Sequence
  Description of Artificial Sequence Synthetic primer
aatgagtcgaaatcattcgttt

SEQ ID NO: 13 - DNA, 21 bp, Artificial Sequence
  Description of Artificial Sequence Synthetic primer
cttagctctcaccatcgctct

SEQ ID NO: 14 - DNA, 24 bp, Artificial Sequence
  Description of Artificial Sequence Synthetic primer
agaataaataggaggcctaggttg

SEQ ID NO: 15 - DNA, 28 bp, Artificial Sequence
  Description of Artificial Sequence Synthetic primer
tattagcaaactcatcactagacatcgt

SEQ ID NO: 16 - DNA, 22 bp, Artificial Sequence
  Description of Artificial Sequence Synthetic primer
tggcaaatacagctcctattga

SEQ ID NO: 17 - DNA, 19 bp, Artificial Sequence
  Description of Artificial Sequence Synthetic primer
aattccccccatccttacc

SEQ ID NO: 18 - DNA, 23 bp, Artificial Sequence
  Description of Artificial Sequence Synthetic primer
gcgacaatggattttacataatg

SEQ ID NO: 19 - DNA, 21 bp, Artificial Sequence
  Description of Artificial Sequence Synthetic primer
aatagccccctttcacttctg

SEQ ID NO: 20 - DNA, 17 bp, Artificial Sequence
  Description of Artificial Sequence Synthetic primer
agaagaagcaggccgga

SEQ ID NO: 21 - DNA, 27 bp, Artificial Sequence
  Description of Artificial Sequence Synthetic primer
ggctaaatcctatatatcttaatggca

SEQ ID NO: 22 - DNA, 22 bp, Artificial Sequence
  Description of Artificial Sequence Synthetic primer
gggaagtagcgtcttgtagacc

SEQ ID NO: 23 - DNA, 19 bp, Artificial Sequence
  Description of Artificial Sequence Synthetic primer
ccctatagcaccccctcta

SEQ ID NO: 24 - DNA, 25 bp, Artificial Sequence
  Description of Artificial Sequence Synthetic primer
ttttagccttctccacttcaagtca

SEQ ID NO: 25 - DNA, 33 bp, Artificial Sequence
  Description of Artificial Sequence Synthetic primer
tttttttttcactccaccttactaccagacaac

SEQ ID NO: 26 - DNA, 37 bp, Artificial Sequence
  Description of Artificial Sequence Synthetic primer
tttttttttttttctactatctcgcacctgaaacaag

SEQ ID NO: 27 - DNA, 43 bp, Artificial Sequence
  Description of Artificial Sequence Synthetic primer
tttttttttttttttttttccataaacaaataggtttggtcct

SEQ ID NO: 28 - DNA, 48 bp, Artificial Sequence
  Description of Artificial Sequence Synthetic primer
tttttttttttttttttttttgagtgactacaaaaaggattagactga

SEQ ID NO: 29 - DNA, 53 bp, Artificial Sequence
  Description of Artificial Sequence Synthetic primer
ttttttttttttttttttttttttttcgttttgtttaaactatataccaattc

SEQ ID NO: 30 - DNA, 50 bp, Artificial Sequence
  Description of Artificial Sequence Synthetic primer
ttttttttttttttttttttttttttttttaggaggcctaggttgaggtt

SEQ ID NO: 31 - DNA, 59 bp, Artificial Sequence
  Description of Artificial Sequence Synthetic primer
tttttttttttttttttttttttttttttttttcctattgataggacatagtggaagtg

SEQ ID NO: 32 - DNA, 68 bp, Artificial Sequence
  Description of Artificial Sequence Synthetic primer
ttttttttttttttttttttttttttttttttttttttttttttttttttcccatccttaccaccctc

SEQ ID NO: 33 - DNA, 72 bp, Artificial Sequence
  Description of Artificial Sequence Synthetic primer
ttttttttttttttttttttttttttttttttttttttttttttttttttttttccagaggttacccaaggc

SEQ ID NO: 34 - DNA, 72 bp, Artificial Sequence
  Description of Artificial Sequence Synthetic primer
ttttttttttttttttttttttttttttttttttttttttttttttttttttatcttaatggcacatgcagc

SEQ ID NO: 35 - DNA, 24 bp, Artificial Sequence
  Description of Artificial Sequence Synthetic primer
tggtcctagcctttctattagctc

SEQ ID NO: 36 - DNA, 24 bp, Artificial Sequence
  Description of Artificial Sequence Synthetic primer
cgtggtgatttagagggtgaactc

SEQ ID NO: 37 - DNA, 23 bp, Artificial Sequence
  Description of Artificial Sequence Synthetic primer
gcgatagaaattgaaacctggcg

SEQ ID NO: 38 - DNA, 25 bp, Artificial Sequence
  Description of Artificial Sequence Synthetic primer
gggttagtccttgctatattatgct

SEQ ID NO: 39 - DNA, 23 bp, Artificial Sequence
  Description of Artificial Sequence Synthetic primer
acgacctcgatgttggatcagga

SEQ ID NO: 40 - DNA, 26 bp, Artificial Sequence
  Description of Artificial Sequence Synthetic primer
ggtctgaactcagatcacgtaggact

SEQ ID NO: 41 - DNA, 24 bp, Artificial Sequence
  Description of Artificial Sequence Synthetic primer
ttccgctacgaccaactcatacac

SEQ ID NO: 42 - DNA, 24 bp, Artificial Sequence
  Description of Artificial Sequence Synthetic primer
gggaatgctggagattgtaatggg

SEQ ID NO: 43 - DNA, 24 bp, Artificial Sequence
  Description of Artificial Sequence Synthetic primer
acagcgctaagctcgcactgattt

SEQ ID NO: 44 - DNA, 24 bp, Artificial Sequence
  Description of Artificial Sequence Synthetic primer
ttgatggcagcttctgtggaacga

SEQ ID NO: 45 - DNA, 28 bp, Artificial Sequence
  Description of Artificial Sequence Synthetic primer
tattagcaaactcatcactagacatcgt

SEQ ID NO: 46 - DNA, 24 bp, Artificial Sequence
  Description of Artificial Sequence Synthetic primer
aaccacagtttcatgcccatcgtc

SEQ ID NO: 47 - DNA, 25 bp, Artificial Sequence
  Description of Artificial Sequence Synthetic primer
ctaagttagctttacagtgggctct

SEQ ID NO: 48 - DNA, 24 bp, Artificial Sequence
  Description of Artificial Sequence Synthetic primer
caacaaccgactaatcaccaccca

SEQ ID NO: 49 - DNA, 24 bp, Artificial Sequence
  Description of Artificial Sequence Synthetic primer
caggttcgtcctttagtgttgtgt

SEQ ID NO: 50 - DNA, 24 bp, Artificial Sequence
  Description of Artificial Sequence Synthetic primer
gccacctactcatgcacctaattg

SEQ ID NO: 51 - DNA, 25 bp, Artificial Sequence
  Description of Artificial Sequence Synthetic primer
agtgtagagggaaggttaatggttg

SEQ ID NO: 52 - DNA, 26 bp, Artificial Sequence
  Description of Artificial Sequence Synthetic primer
cctgaacgcaggcacatacttcctat

SEQ ID NO: 53 - DNA, 24 bp, Artificial Sequence
  Description of Artificial Sequence Synthetic primer
tgagcctagggtgttgtgagtgta

SEQ ID NO: 54 - DNA, 22 bp, Artificial Sequence
  Description of Artificial Sequence Synthetic primer
cagctatccattggtcttaggc

SEQ ID NO: 55 - DNA, 22 bp, Artificial Sequence
  Description of Artificial Sequence Synthetic primer
ttaacgagggtggtaaggatgg

SEQ ID NO: 56 - DNA, 24 bp, Artificial Sequence
  Description of Artificial Sequence Synthetic primer
accctaacaggtcaacctcgcttc

SEQ ID NO: 57 - DNA, 24 bp, Artificial Sequence
  Description of Artificial Sequence Synthetic primer
agaaatcctgcgaataggcttccg

SEQ ID NO: 58 - DNA, 24 bp, Artificial Sequence
  Description of Artificial Sequence Synthetic primer
caactacaagaacaccaatgaccc

SEQ ID NO: 59 - DNA, 24 bp, Artificial Sequence
  Description of Artificial Sequence Synthetic primer
tcatcatgcggagatgttggatgg

SEQ ID NO: 60 - DNA, 24 bp, Artificial Sequence
  Description of Artificial Sequence Synthetic primer
actcaaatgggcctgtccttgtag

SEQ ID NO: 61 - DNA, 25 bp, Artificial Sequence
  Description of Artificial Sequence Synthetic primer
catctccggtttacaagactggtgt

SEQ ID NO: 62 - DNA, 17 bp, Artificial Sequence
  Description of Artificial Sequence Synthetic primer
gaataggcttccggctg

SEQ ID NO: 63 - DNA, 24 bp, Artificial Sequence
  Description of Artificial Sequence Synthetic primer
gcaagggaaagatgaaaaattata

SEQ ID NO: 64 - DNA, 29 bp, Artificial Sequence
  Description of Artificial Sequence Synthetic primer
ttttacactactataaccaccctaaccct

SEQ ID NO: 65 - DNA, 30 bp, Artificial Sequence
  Description of Artificial Sequence Synthetic primer
tttttttttttttaatagcggctgcaccat

SEQ ID NO: 66 - DNA, 32 bp, Artificial Sequence
  Description of Artificial Sequence Synthetic primer
tttttttttttttttggagttgcaccaaaatt

SEQ ID NO: 67 - DNA, 41 bp, Artificial Sequence
  Description of Artificial Sequence Synthetic primer
tttttttttttttttttttttttacacatgcaagcatcccc

SEQ ID NO: 68 - DNA, 42 bp, Artificial Sequence
  Description of Artificial Sequence Synthetic primer
ttttttttttttttttttttggtgctatagggtaaatacggg

SEQ ID NO: 69 - DNA, 48 bp, Artificial Sequence
  Description of Artificial Sequence Synthetic primer
ttttttttttttttttttttttttttttggttgatattgctagggtgg

SEQ ID NO: 70 - DNA, 53 bp, Artificial Sequence
  Description of Artificial Sequence Synthetic primer
tttttttttttttttttttttttttttttttgggcctgtccttgtagtataaa

SEQ ID NO: 71 - DNA, 58 bp, Artificial Sequence
  Description of Artificial Sequence Synthetic primer
tttttttttttttttttttttttttttttttttttcctagggtgttgtgagtgtaaat

SEQ ID NO: 72 - DNA, 63 bp, Artificial Sequence
  Description of Artificial Sequence Synthetic primer
ttttttttttttttttttttttttttttttttttttttgagattgtaatgggtatggagacat

SEQ ID NO: 73 - DNA, 68 bp, Artificial Sequence
  Description of Artificial Sequence Synthetic primer
ttttttttttttttttttttttttttttttttttttttttttttggttagaactggaataaaagctag

SEQ ID NO: 74 - DNA, 73 bp, Artificial Sequence
  Description of Artificial Sequence Synthetic primer
tttttttttttttttttttttttttttttttttttttttttttttttcgtcctttagtgttgtgtatggttat

SEQ ID NO: 75 - DNA, 78 bp, Artificial Sequence
  Description of Artificial Sequence Synthetic primer
tttttttttttttttttttttttttttttttttttttttttttttttttttttttttttttggtggggaggtcgatga

SEQ ID NO: 76 - DNA, 83 bp, Artificial Sequence
  Description of Artificial Sequence Synthetic primer
ttttttttttttttttttttttttttttttttttttttttttttttttttttttttttttttttatgaccccaatacgcaaaa